(19) United States
(12) Patent Application Publication
(10) Pub. No.: US 2015/0157202 A1
(43) Pub. Date: Jun. 11, 2015

(54) Method and System for Optic Nerve Head Shape Quantification

(71) Applicant: Alexander Ulrich Brandt, Berlin (DE)

(72) Inventors: Alexander Ulrich Brandt, Berlin (DE); Ella Maria Kadas, Berlin (DE); Friedemann Paul, Berlin (DE); Konrad Polthier, Berlin (DE)

(21) Appl. No.: 14/100,161

(22) Filed: Dec. 9, 2013

(51) Int. Cl. A61B 3/10

(52) U.S. Cl. CPC A61B 3/102

(57) ABSTRACT

A fully automated optic nerve head description system based on optical coherence tomography that works with heavily deformed nerve heads and allows the generation of parameters describing swelling, including correlation with intracranial pressure both in detection and progression.

This application claims the benefit of the filing date under 35 U.S.C. §119(e) of Provisional U.S. Patent Application Ser. No. 61/735,092, filed on Dec. 10, 2012, which is hereby incorporated by reference in its entirety.

### BACKGROUND OF THE INVENTION

Optical coherence tomography (OCT) is a non-invasive imaging method, which creates in vivo cross-sectional patterns of the retina. In recent years, OCT has become a valuable tool for assessing retinal axonal damage in several neurological diseases such as optic neuritis, multiple sclerosis, neuromyelitis optica, spinocerebellar ataxia, and Parkinson's disease. Moreover, retinal changes measured with OCT are linked both with morphologic and metabolic changes in the brain, thus providing an easily accessible window into the brain in neurologic diseases.

Modern spectral domain OCT allows for non-invasive, spatial imaging of retinal layers using manual or semi-automatic segmentation techniques. Some of these segmentation methods are included in current devices others are available externally. All existing methods are limited by their reliance on specific retinal layouts in order to work correctly. In particular, current automatic segmentation methods are unable to process complex structures like the optic nerve head (ONH) in diseases that show profound ONH alterations (i.e. swelling) like, for example, idiopathic intracranial hypertension (IIH) or optic neuritis (ON). FIG. 1 is a schematic diagram of the human eye anatomy that shows on the left a normal eye and on the right schematically an ONH swelling.

The most common factors leading to pathologic ONH swelling (also called papilledema) are, for example, inflammation of the optic nerve (e.g. in multiple sclerosis or neuromyelitis optica) or increased intracranial pressure (e.g., ICP, such as in IIH or brain cancer). Blood vessel pathologies in and around the optic nerve can also show specific alterations, including swelling.

Existing methods focus on detection of ONH flattening or degeneration in diseases such as glaucoma. In these cases, 3D spatial conformation changes (e.g. comparison of focal sector changes in the nasal, temporal, superior and inferior quadrants of the ONH) are used for diagnosis. These methods are inapplicable in pathologies with an increased ONH, because the swelling obscures the specialized segmentation routines.

Due to the inability of current methods to measure papilledema directly, previous clinical studies have used a commonly available method using the retinal nerve fiber layer thickness (RNFLT) in a peripapillary ring scan. This only allows for inadequate measurement of ONH swelling, for example, lowering greatly sensitivity and specificity of the measurement. Additionally, only severe swellings that protrude into the measured area can be detected whereas more moderate swellings are overlooked.

With these limitations, ONH swelling quantification to measure e.g. optic nerve inflammation or intracranial pressure changes is not possible. To measure optic nerve inflammation, only suboptimal measures like RNFLT are available. To measure ICP, the clinician is forced to rely on clinical symptoms and direct measurement of ICP by lumbar puncture for monitoring treatment effects and disease progression.

The above information disclosed in this Background section is only for enhancement of understanding of the background of the present invention and therefore it may contain information that does not form the prior art that is already known in this country to a person of ordinary skill in the art.

### SUMMARY OF THE INVENTION

A method and system was developed, herein described by exemplary embodiments, that is useful, by way of example, in (a) detecting ONH swelling, (b) implementing a robust reference for ONH quantification, and (c) indirectly measuring intracranial pressure levels and changes in patients.

An exemplary embodiment of the present invention includes a reliable clinical method of detecting and reconstructing outer retinal reference layers utilizing minimal information, thus making it applicable in conditions where layers are barely detectable in OCT. Information about optic nerve inflammation and intracranial pressure may be derived and ONH shape may be quantified in conditions when the ONH might be highly deformed, for example, in multiple sclerosis, neuromyelitis optica, optic neuritis, idiopathic intracranial hypertension, brain tumors, vascular disorders of the eye or optic nerve, normal pressure hydrocephalus or other diseases.

In this exemplary embodiment the method and system may include some or all of the following steps (a) performing or providing a 3D OCT scan of the ONH of one or both eyes of a subject, (b) detecting limiting retinal layer fragments from OCT B-scans with a minimum set of available information, (c) enhancing and extending the fragments to full boundaries for ONH shape assessment, (d) using the boundaries to calculate and describe the ONH in one B-scan or a 3D reconstruction using several B-scans, (e) calibrating the ONH shape data with ICP measurements and (f) using the ONH shape to detect differences between different conditions e.g. for diagnosis or to detect changes over time in patients, e.g. for monitoring disease progression or therapy efficacy.

### BRIEF DESCRIPTION OF THE DRAWINGS

FIG. 1 is a diagram that shows the normal eye anatomy on the left and a schematic representation of the ONH condition during swelling due to e.g. inflammation or ICP elevation on the right.

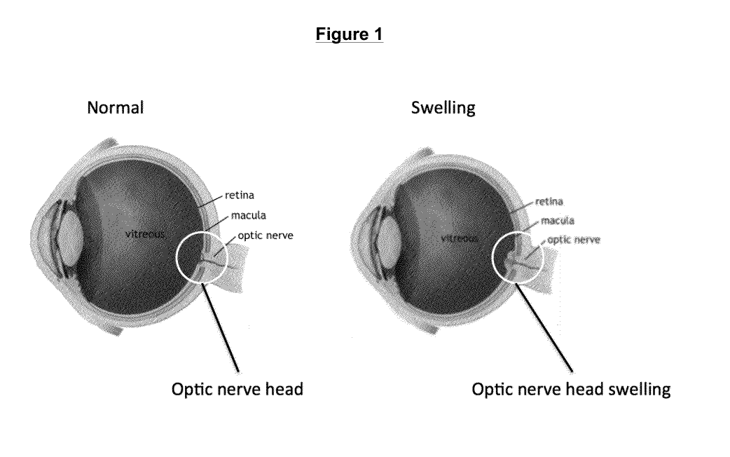

FIG. 2 is a schematic diagram of an OCT B-Scan that shows the OCT detectable layers and membranes of the retina.

FIG. 3 is a schematic diagram that shows ONH B-scans from different patients at different positions relative to the ONH and elucidates different changes that occur in this region.

FIG. 4 is a block diagram of a sample workflow for detecting RPE and calculating ONH volume and height.

FIG. 5 is a block diagram that shows the strategy for initial RPE detection and extension. (a) Detecting the possible upper boundary, (b) detecting the possible lower boundary, and (c) growing RPE from initial seed points.

FIG. 6 is a block diagram that shows the diagnostic workflow incorporating an exemplary embodiment of the invention in diagnosis of papilledema.

FIG. 7 is a block diagram that shows the diagnostic workflow incorporating the invention in diagnosis of ICP.

FIG. 8 is a table that shows sample data from a study investigating ONH shape differences between IIH patients and controls. (a) The 3D representation of an ONH from a healthy control eye, (b) the 3D representation of an ONH from a patient with IIH, (c) differences between healthy control and IIH patient eyes in ONHV and (d) difference in ONHV between treated and untreated IIH patients.

FIG. 9 is a block diagram that shows the diagnostic workflow incorporating the invention in diagnosis of real-time ICP changes.

FIG. 10 is a table that shows sample data from a study investigating real-time changes in ONH shape during willful ICP manipulation from a patient with implanted ICP sensor. (a) shows the repeatedly measured B-scan of the ONH, (b) shows the extracted area analyzed, (c) shows area size changes over time without pressure maneuver, (d) shows area size changes over time with pressure maneuver.

### DETAILED DESCRIPTION OF THE INVENTION

In the following detailed description, only exemplary embodiments of the present invention have been shown and described, simply by way of illustration. As those skilled in the art would realize, the described embodiments may be modified in various ways, all without departing from the spirit or scope of the present invention. Accordingly, the drawings and description are to be regarded as illustrative in nature and not restrictive.

### ABBREVIATIONS

The following abbreviations are used throughout the document: OCT—optical coherence tomography; ONH—optic nerve head; IIH—idiopathic intracranial hypertension; ON—optic neuritis; NPH—normal pressure hydrocephalus; ONHH—optic nerve head height; ONHV—optic nerve head volume; RPE—retinal pigment epithelium; ILM—inner limiting membrane; MS—multiple sclerosis; ICP—intracranial pressure

### ONH Shape Quantification

In an embodiment of the present invention, an automated method for segmenting retinal reference layers or lines from spatial high-resolution OCT ONH scans is provided. A number of B-scans are recorded from one or both eyes of a patient to assemble the ONH. Reference lines or layers are detected on every B-scan. The reference lines or layers are used to calculate the ONH shape and apply it for characterizing ONH swelling and deformation.

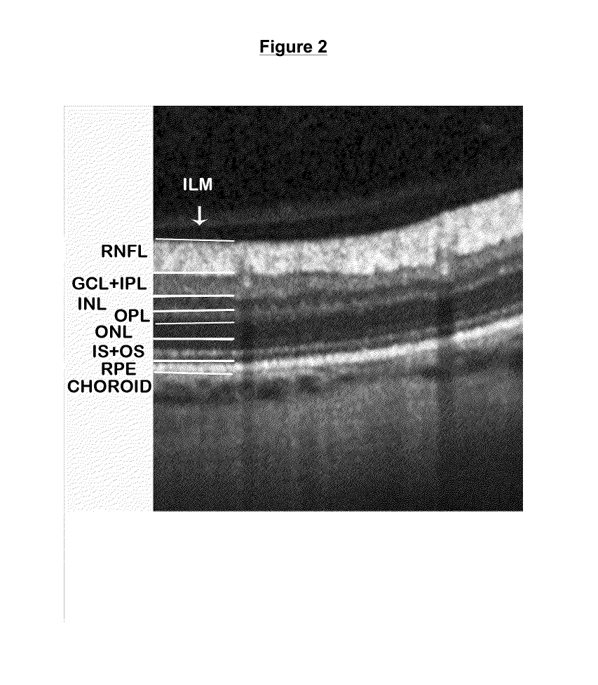

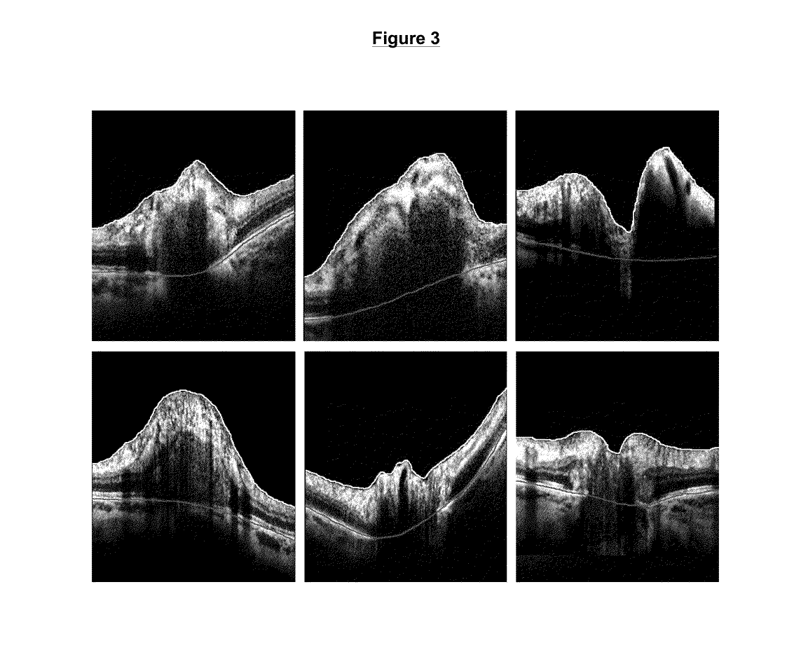

In another embodiment of the present invention, the method detects and reconstructs the outer retinal reference layers (in one aspect, the retinal pigment epithelium, RPE) with minimal information, thus making it applicable in conditions where layers are barely detectable in OCT, i.e., when the ONH is swollen. Retinal layers are shown in FIG. 2. In swollen ONH, OCT scans tend to have regions of strong varying intensity values caused by the edema. Additionally, scans are characterized by an increased intrinsic speckle noise making a reliable differentiation of intraretinal layers challenging to impossible. Different swollen or deformed ONH configurations are shown in FIG. 3.

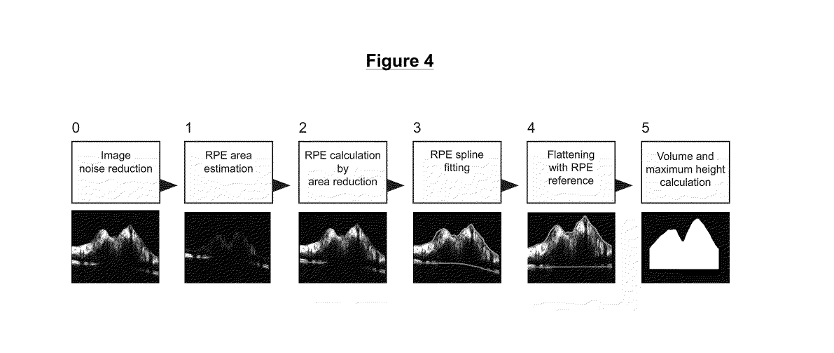

In an exemplary embodiment of the present invention, a 3D ONH scan was performed with 145 slices (B-scans), focusing the optic nerve head with a scanning angle of 15°×15° and a resolution of 384 A-scans per B-scan. The volume (ONHV) and maximum height (ONHH) of the disc edema are measured and determined. Two reference boundaries, i.e. the inner limiting membrane (ILM) and a hypothetical extension of the peripapillary retinal pigment epithelium (RPE) through the ONH, are determined on each B-scan. The edema is then defined as the area enclosed by these two layers. The ILM is provided in sufficient quality. The method, for example, focuses on segmenting the RPE to create a base area for further calculation of both parameters to compute the volume and maximal height of the edema. First an initial region that contains this layer is estimated. Starting from this region, unnecessary pixels are discarded. The RPE curve, describing the layer, is obtained by a fully automatic least square spline fitting of 2nd or 3rd order depending on the number of detected RPE segments. Further, for example, an image flattening on each slice using again the RPE, followed by the final volume and height computation is used to account for the natural retinal curvature seen in OCT images. For the volume measurement a threshold of 20 pixels was applied from the reference height computed at the right side and left side of each flattened B-scan. The area of the edema found on each B-scan multiplied by the spatial spacing is added to obtain the final volume. This threshold was selected to include most of the ONH and to provide a satisfying volume of the swelling in IIH as well as healthy controls. The workflow of this embodiment is shown in FIG. 4: 0) First, OCT B scans are cleaned from noise and smoothed; 1) then, the area in which the RPE is expected is narrowed by removing bright upper layers of the scan, resulting in the brightest areas belonging most likely to the RPE; 2) From the A scan at the first quarter and the A scan at the last quarter of the B scan, the possible RPE area is further reduced by detecting the brightest areas of the image; 3) On the proposed RPE candidates, a least square spline approximation is applied, resulting in a hypothetical RPE through the ONH; the ILM is provided; 4) The scans are flattened using the RPE as reference; 5) Finally, volume and height are calculated in the newly defined area. The method and system may be implemented in computer software.

In another illustrative example, the volume and the height of the ONH edema is calculated using B-scans and corresponding ILM positions. The ILM information is provided. The outer retinal layer (i.e. the RPE), for example, is detected around the ONH and extended through the ONH as a theoretical lower bound to volume and height measurements. The RPE separates the other layers from the choroid (FIG. 2).

### Step 1: Preprocessing

In another example of the present invention, a B-scan from the 3D OCT scans, the intrinsic speckle noise is reduced by denoising each B-scan using anisotropic diffusion. The iteration step is set to 10. Next a relative homogeneous region is created from the corresponding ILM to the inner nuclear layer (INL) by dilating the ILM. The disc structure radius is set to 4 in this step. After that, each B-scan is smoothed with a large Gaussian filter. In this example filter size is set to [7,7], with σ=5. The resulting image contains three regions in the following order from top to bottom: light grey, black and grey.

### Step 2: RPE Region

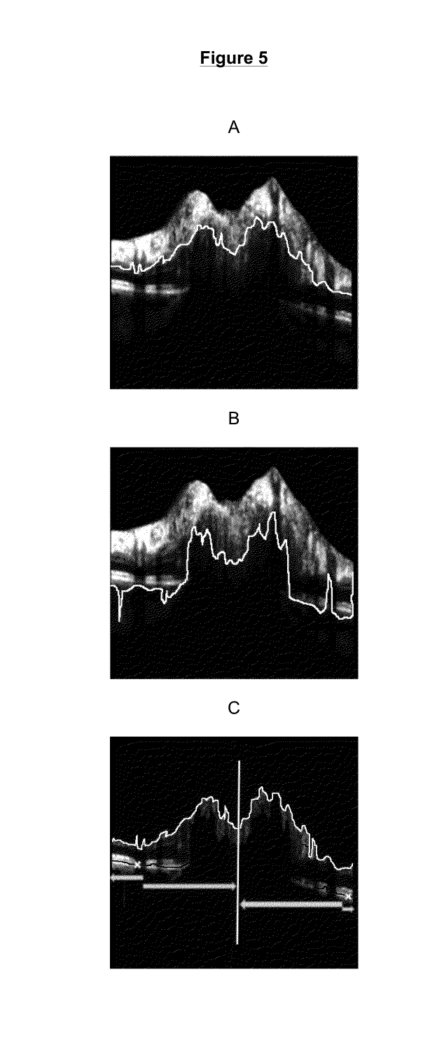

Two curves, for example, are detected to bound the search area for the RPE from the two lower regions (FIGS. 5 a and 5 b). The candidate pixels for the construction of the curves are found per column
for the upper one from top to bottom, starting at the ILM:

{p(xᵢ,yⱼ)|I(p(xᵢ,yⱼ)<60,0<i<m,ILM(i)<j<n}
for the lower one from bottom to top:

{p(xᵢ,yⱼ)|I(p(xᵢ,yⱼ)<20,0<i<m,ILM(i)<j<n,}

where m×n is the total number of pixels in each B-scan, and I(p) the intensity value at a point p. The RPE regions with low intensity values may also be taken into account. Fitting a cubic polynomial to the set of top and lower resulting pixels, two boundary curves C1 (FIG. 5-(a)) and C2 are created (FIG. 5-(b)).

### Step 3: RPE Initial Pixels

Using the two curves C1 and C2 from the previous step, in one example, RPE pixels candidates within the bounded region are now chosen. RPE consistently shows pixels having the highest intensity value among all other layers. This may result in strong artifacts due to the ONH swelling. Therefore, and to ensure a spatial choice that respects the anatomical position of this layer, information about pixel intensity and position in the grey value profile of each column in a B-scan may be used. For each profile, the set P of peaks is detected. From each of these sets, for example, a point p with d(p, C1)>20px is added to a list L which meets the conditions:

$$\{ \begin{matrix}p = min {M}_{h ( p ) ∈ M} , & M = \{ x , \vert h ( x ) - h ( s ) \vert < 10 , ∀ s ∈ P \} \\ p = max {M}_{h ( p ) ∈ M} , & M = \{ x , \vert h ( x ) - h ( s ) \vert > 10 , ∀ s ∈ P \}\end{matrix}$$

where d(p,C1) represents the distance from the candidate point p to the corresponding point of the upper bounding curve C1, and h(p) is the grey value of a point in the intensity profile.

From the list L of each B-scan, the final selection of points to create the curve, describing the RPE layer, is constructed. Outliers might still be present in L in B-scans that contain the region of the edema. Outliers are accounted for by focusing on the RPE information at the outer left and right side of each scan. Two lists L1 and L2 are created from pixels in L. For each side a point p(xᵢ,yᵢ) in the first quarter from left and right of the scan with minimal yᵢ coordinate is detected. These give the starting reference height for creating two lists. Starting from these seed points to the right and left, pixels are added iteratively to the corresponding list if they meet the following conditions (FIG. 5 (c)):

$$\{ \begin{matrix}\vert {x}_{i} - {x}_{i - 1} \vert < 5 px , & for \vert {y}_{i} - {y}_{i - 1} \vert < 5 px \\ \vert {x}_{i} - {x}_{i - 1} \vert < 10 px , & for \vert {y}_{i} - {y}_{i - 1} \vert < 15 px\end{matrix}$$

In case of missing image information, RPE segmentation data from the previous scan is taken into account.

### Step 4: RPE Curve

Once the two lists are created, a curve fitting is applied. In one example, a least square spline approximation to L1∪L2 is performed, with knots and order of the spline, quadratic or cubic, depending on the number of pixels of L1, compared to L2. The scan alignment step, for example, is performed using column shifting.

### Step 5: ONH Volume and Height

For the volume measurement, a threshold number of pixels can be applied from the reference height computed at the right side and left side of each B-scan. The areas found on each B-scan, multiplied by the spatial spacing (e.g., how many pixels represent how many micrometers) were added to obtain the final volume. The threshold, for example, is 20 pixels.

### EXAMPLES

The method can be used to quantify ONH shape even in conditions when the ONH might be highly deformed, like, for example, in multiple sclerosis, neuromyelitis optica, optic neuritis, idiopathic intracranial hypertension, brain tumors, vascular disorders of the eye or optic nerve, normal pressure hydrocephalus or other diseases.

### Example

### Measurement of Swelling During an Acute Optic Neuritis or Vascular Event

In an exemplary embodiment of the present invention, swelling or flattening is quantified during an acute inflammation, e.g. an optic neuritis, both in single measurements (e.g. swollen/flattened ONH in comparison to a reference group (e.g. a reference database from healthy controls) and follow up measurements (e.g. ONH swelling or flattening over a period of time or in response to treatment in a single patient).

Further, swelling or flattening is quantified, for example, during an acute or chronic vascular event, e.g. an embolism of the central retinal artery, both in single measurements (e.g. swollen/flattened ONH in comparison to a reference group (e.g. a reference database from healthy controls) and follow up measurements (e.g. ONH swelling or flattening over a period of time or in response to treatment in a single patient).

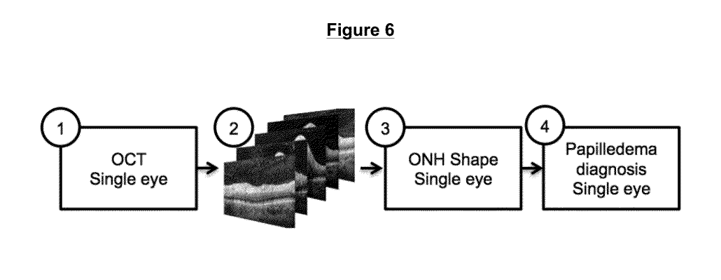

FIG. 6 shows the workflow in this example: (1) an eye is measured using OCT; (2) an ONH volume scan is taken consisting of several B-scans over the ONH; (3) the methods as described above are applied resulting in ONH shape data like ONHV and ONHH; and (4) this data is then used to aid in diagnosis of ONH swelling, for example, in acute swelling (with comparison against, i.e., a normative database from healthy controls) or longitudinally to follow changes in one eye over a period of time using repeated scans and method applications.

### Example

### ICP Changes

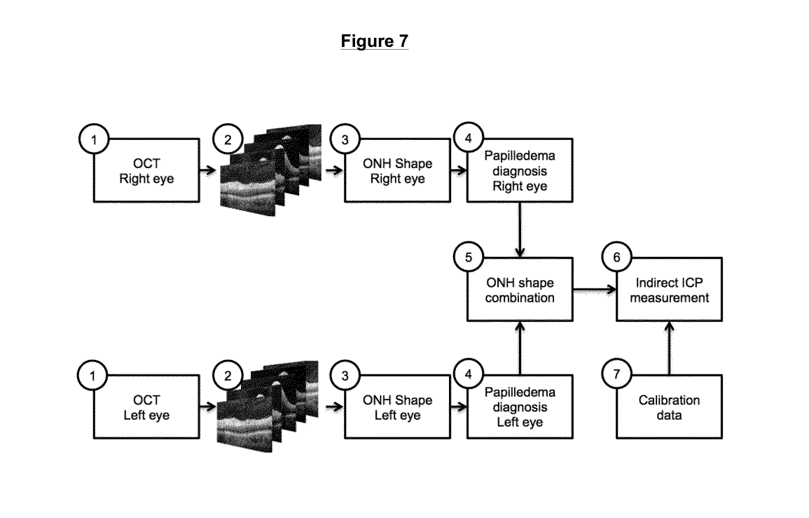

In another example, papilledema is accessed in conditions with elevated intracranial pressure (ICP). Patients with ICP elevation like in idiopathic intracranial hypertension (IIH) most often present papilledema at the ONH, associated with visual field losses. Quantifying ONH edema in IIH and other diseases with altered ICP is important for diagnosis and for monitoring progression and treatment effectiveness. ONH edema may be quantified for indirect measurement of ICP levels and changes. For example, derived shape values (like ONH volume and height) are used to measure ICP changes. The ONH shape information from one or both eyes, alone or in combination, is used to describe ICP changes and to evaluate single measurements (e.g. elevated ICP in comparison to a reference group (e.g. a reference database from healthy controls)) as well as follow up measurements (e.g. changes in ICP over a period of time or in response to treatment in a single patient). For example, the correlation between ICP and ONH changes is calibrated. FIG. 7 shows the workflow for this example: (1) OCT is performed both in the right and left eye of a patient; (2) and an ONH volume scan in each eye is taken; (3) these scans are then analyzed using the methods described above; (4) aiding in diagnosis and quantification of papilledema; (5) in a next step, the data from both eyes is combined (e.g., using the mean from both eyes, or in other examples, weighting one eye stronger than the other, or weighting the one eye with stronger edema stronger); (6) this combined data is then used to indirectly predict ICP in the patients. This can be used, for example, for primary diagnosis or for following treatment efficacy or progression in patients already diagnosed; and (7) the validity of this can be either supported by calibration data from cross-sectional studies comparing actual ICP with ONH shape data or with specimen specific data from a single patient for longitudinal investigations.

In a cross-sectional pilot trial comparing 19 IIH patients and controls matched for gender, age and body mass index, each participant underwent OCT. ONH volume (ONHV) and ONH height (ONHH) were quantified in accordance with an embodiment of the present invention. Peripapillary RNFLT did not show differences between controls and IIH patients. The ONHV and ONHH, however, were distinguishable between controls, treated and untreated patients. Both ONHV and ONHH measurements were correlated to levels of ICP.

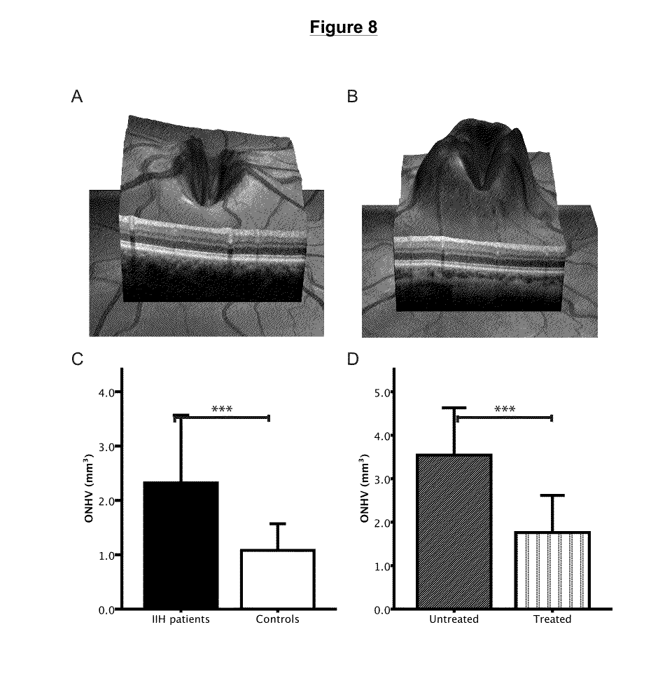

FIG. 8 shows the summary from this study: A) 3D spectral domain OCT ONH measurement from a matched control ONH; B) 3D spectral domain OCT ONH measurement from an IIH patient with a diagnosed papilledema; C) groups differences in optic nerve head volume (ONHV) between IIH patients (black bar) and controls (white bar); and D) group difference in ONHV between medically untreated (gray bar) and treated (vertical lines bar) IIH patients. Error bars represent 1× standard deviation in figures c and d. ***=p<0.001 from Generalized Estimating Equations Models.

In another example, papilledema is assessed in other diseases with elevated ICP. E.g. ICP changes could be monitored using the invention in acute stroke, brain hemorrhage, brain tumors or other conditions with changing ICP.

### Automatic Versus Manual Segmentation Validation Study.

Ten randomly selected healthy controls and five IIH patients were used. In total, scans from 20 eyes, each with 145 B-scans, were manually segmented by two expert graders and compared to the results of automatic segmentation as described in this invention. The results in Table 1 show that the RPE is accurately detected in healthy control scans as well as in scans from IIH patients. The quantitative results indicate a very good statistical agreement and high correlation between the manual graders and the presented automated method in accordance with an embodiment of the present invention.

TABLE 1
Differences in RPE detection for 1450 B-scans of healthy controls
and 1450 B-scans of IIH patients, between first expert manual
grader compared to the proposed method (Column I), between
the second expert manual grader compared to the proposed method
(Column II). Column III reports the differences between the
two manual graders. Each pixel is 3.8717 μm.

| | | | Manual Grader |
|---|---|---|---|
| | Manual Grader | Manual Grader | 1 vs. Manual |
| RPE Differences | 1 vs. Method | 2 vs. Method | Grader 2 |
| Healthy controls. | | | |
| Mean Difference | 1.1515 | 1.2265 | 1.1284 |
| Standard deviation | 0.1628 | 0.1813 | 0.1725 |
| intraclass correlation | 0.9996 | 0.9999 | 0.9998 |
| coeff. | | | |
| IIH patients. | | | |
| Mean Difference | 1.3478 | 1.4093 | 1.3859 |
| Standard deviation | 0.3724 | 0.392 | 0.3578 |
| intraclass correlation | 0.9979 | 0.9983 | 0.9998 |
| coeff. | | | |

### Example

### Measurement of Real-Time ICP Changes

Further, by way of example, short-term changes in ICP are derived. For this, recorded B-scans are limited in number and ONH shape is derived from only few (as many as one) B-scan(s) to achieve a high time resolution. In this case, the ONH measurement can include less than the full ONH and be limited to a smaller area. B-scans are then recorded continuously over a time period and the changes in ONH shape (e.g. volume, area or height) are described over time.

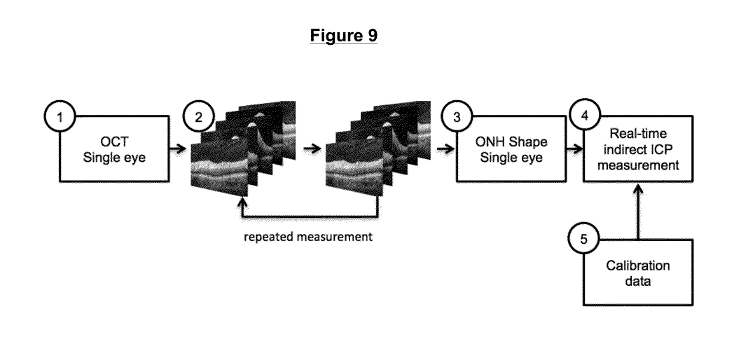

FIG. 9 shows the workflow for this example: (1) OCT is taken from a single eye; (2) a defined region over the ONH is repeatedly measured over time; (3) ONH shape data is calculated using the described methods; (4) from these data over time real-time changes in ICP can be predicted; and (5) the validity of this may be supported by calibration data from cross-sectional studies comparing actual ICP with ONH shape data, or with specimen specific data from a single patient for longitudinal investigations.

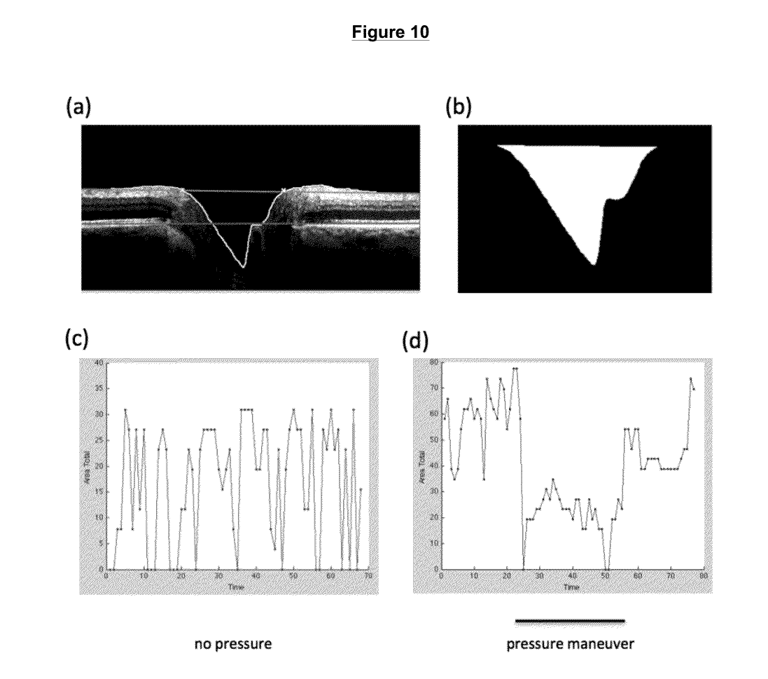

FIG. 10 shows sample data from a clinical study investigating a patient with implanted ICP sensor and able to willingly increase ICP: (a) shows a central B-scan that was measured in this patient over a time of several minutes every second; (b) shows the extracted area using the methods described above; (c) shows the area changes over time without pressure maneuver; and (d) shows the drop in area size (meaning protrusion of the ONH) when the patient applied the willful pressure maneuver.

We claim:

1. A method for describing the optic nerve head using structural information comprising: (A) providing a tomographic scan of the optic nerve head area of the eye; (B) defining two or more boundaries, wherein a boundary is the inner limiting membrane and a boundary is the retinal pigment epithelium or basal membrane; (C) interpolating the retinal pigment epithelium or basal membrane through the optic nerve head; (D) constructing volume and shape information between the boundaries.

2. The method as recited in claim 1 wherein papilledema or optic nerve head swelling is quantified.

3. The method as recited in claim 1 wherein intracranial pressure is described.

4. The method as recited in claim 1 wherein dynamic changes of the optic nerve head are described.

5. The method as recited in claim 1 wherein changes in optic nerve head volume and shape are calibrated using optic nerve head information from one or more individuals at one time point or over time.

6. The method as recited in claim 1 wherein changes in optic nerve head volume and shape are calibrated using additional medical information from one or more individuals at one time point or over time.

7. A system for describing the optic nerve head using structural information comprising an information processing apparatus that executes the method as recited in claim 1.

8. A system as recited in claim 7 wherein the information process apparatus is part of an imaging instrument.
